(19) United States
(12) Patent Application Publication
(10) Pub. No.: US 2011/0039793 A1
(43) Pub. Date: Feb. 17, 2011

(54) Application of ginsenoside Rb1 in preparing medicaments for treating dilated cardiomyopathy

(76) Inventors: Lianfeng Zhang, Beijing (CN); Haiping Zhao, Beijing (CN); Chuan Qin, Beijing (CN); Wanbo Li, Beijing (CN)

Correspondence Address: ZHEN ZHENG LU, 1730 HUNTINGTON DRIVE #304, DUARTE, CA, 91010, US

(21) Appl. No.: 12/921,397

(22) PCT Filed: Sep. 18, 2008
(86) PCT No.: PCT/CN08/72409

§ 371 Date: Sep. 7, 2010

(30) Foreign Application Priority Data
Jul. 7, 2008 (CN) 200810116202.7

(51) Int. Cl. A61K 31/704; C07J 17/00; A61P 9/00

(52) U.S. Cl. 514/26; 536/5

(57) ABSTRACT

A method of preparing medicaments for treating dilated cardiomyopathy includes the step of applying ginsenoside Rb1. Long-term treatment of ginsenoside Rb1 can reduce the mortality rate, improve the cardiac function, reduce the dilation of the left ventricular, reduce the hypertrophy of myocytes and the proliferation of collagen, reduce the uneven thickness of myocytes, mitochondrial swelling, break of crista, sarcoplasmic reticulum expansion and destruction of intercalated disc connection, increase the expression of connexin Cx40, E-cadherin and P-cadherin, fibroblast growth factor FGF2, FGF7, FGF8, FGF10, and reduce the expression of integrin Itga8 and Itgbp.

### BACKGROUND OF THE PRESENT INVENTION

1. Field of Invention

The present invention relates to a medical field, and particularly to an application of ginsenoside Rb1 in preparing medicaments for treating dilated cardiomyopathy.

2. Description of Related Arts

Dilated cardiomyopathy is a myocardial disease with the main features of ventricular dilation and ventricular systolic function impairment. Its clinical characteristics are progressive heart failure, arrhythmia, thromboembolism, and even sudden death. And its five-year survival rate is less than 50%. Its typical pathological features are ventricular dilation, thin ventricular wall, myocardial apoptosis, and the main causes are viral myocarditis and gene mutation which are accounting for 70% of the incidence of the dilated cardiomyopathy.

Clinical studies have found a variety of mutant genes associated with dilated cardiomyopathy, most gene mutations will lead to HCM and DCM simultaneously, however, the R141W mutation of cTnT protein (arginine (Arg)→tryptophan (Trp)) lead to DCM. Therefore, the transgenic model of the R141W mutation has a specificity for studying the pathogenesis of dilated cardiomyopathy. The cTnT^{R141W} transgenic dilated cardiomyopathy mice model, established in Genetic Center, Institute of Laboratory Animal, Chinese Academy of Medical Sciences, is a good genetically engineer animal model of dilated cardiomyopathy. The mice model of cTnT^{R141W} shows dilated ventricular, thin ventricular wall and decreased cardiac function, which are similar to pathological manifestations of human dilated cardiomyopathy.

Ginsenoside Rb1 (C₅₄H₉₂O₂₃.H₂O) is known as a monomer existing in a certain number of plants, such as Ginseng, and its structure and related biological activities have been reported in the related textbooks and published literatures. However, the application of ginsenoside Rb1 in preparing medicaments for treating family dilated cardiomyopathy has not yet been reported.

### SUMMARY OF THE PRESENT INVENTION

The object of the present invention is to provide an application of ginsenoside Rb1 in preparing medicaments for treating dilated cardiomyopathy.

To accomplish the object, the present invention provides a method of preparing medicaments for treating dilated cardiomyopathy comprising the step of applying ginsenoside Rb1.

The ginsenoside Rb1 comprises ginsenoside Rb1 monomer and excipient or vector.

The dilated cardiomyopathy is the death caused by cTnT^{R141W} (arginine Arg of troponin T→tryptophan Trp).

The dilated cardiomyopathy is the progressive dilation of left ventricular and decrease of cardiac function caused by cTnT^{R141W}.

The dilated cardiomyopathy is the irregular hypertrophy of left ventricular myocytes and the proliferation of interstitial collagen caused by cTnT^{R141W}.

The dilated cardiomyopathy is the uneven thickness and disorderly arrangement of myocytes, swelling mitochondria, break of crista, expanded sarcoplasmic reticulum and destruction of intercalated disc connection caused by cTnT^{R141W}.

The dilated cardiomyopathy is the decreased expression of connexin Cx40, E-cadherin and P-cadherin and the increased expression of, Itga8 and Itgbp caused by cTnT^{R141W}.

The dilated cardiomyopathy is the decreased expression of fibroblast growth factor FGF2, FGF7, FGF8 and FGF10 caused by cTnT^{R141W}.

Ginsenoside Rb1 of the present invention is generally used in the form of drug combination or the monomer. It can be administered orally or injectedly, and can also be made into tablet, capsule, powder, granule, pastille or oral liquid for clinical application. The above mentioned medicaments in various forms are prepared in accordance with the conventional methods of the pharmaceutical field.

Ginsenoside Rb1 of the present invention has the functions followed. It can lessen the progressive dilation of left ventricular and decreased cardiac function which are caused by troponin mutation, attenuate the irregular hypertrophy of myocardial cells, reduce the formation of interstitial collagen fibers, improve the arrangement of myocardial fibers and intercalated disc structure, reduce mitochondria swelling and sarcoplasmic reticulum expansion, increase the expression of myocardial connexin, and reduce the expression of integrins and cadherins. Its mechanism is associated with the expression of the regulation growth factor. Therefore, direct and strong experimental evidence and theoretical basis are provided for the application of ginsenoside Rb1 in the treatment of dilated cardiomyopathy. The present invention exhibits the development of application value.

The objective, features and advantages of the present invention will become apparent from the following detailed description, the drawings and together with the appended claims.

### BRIEF DESCRIPTION OF THE DRAWINGS

FIG. 1 is an electrophoretogram of PCR products according to a first preferred embodiment of the present invention.

FIG. 2 is the survival analysis diagram of the above first preferred embodiment of the present invention.

FIG. 3 is an echocardiography data according to a second preferred embodiment of the present invention.

FIG. 4 is an electron micrograph according to a fourth preferred embodiment of the present invention.

FIG. 5 is an electrophoretogram of RT-PCR according to a fifth preferred embodiment of the present invention.

### DETAILED DESCRIPTION OF THE PREFERRED EMBODIMENT

The present invention is further explained with the accompany embodiments and drawings.

### Example 1

Effects on the survival rate of the mice model with dilated cardiomyopathy after a long-term administration of ginsenoside Rb1.

1. The PCR Detection of cTnT^{R141W} Transgenic Mice

1) Scissor 0.5-1 cm mice tail and put it into an EP tube, add 0.5 ml mice tail lysate into the EP tube, then add 10 μl protease K (20 mg/ml), and shake overnight at 55° C.;

2) The next day, add 300 μl saturated sodium chloride, quietly placed on ice for 15 min, and centrifugate at 13000 rpm for 15 min;

3) Remove 600 μl supernatant, and add the same volume of isopropanol into the supernatant, mix reversedly till flocculent deposition appears and centrifugate at 13000 rpm for 15 min;

4) Wash the deposition once with 70% ethanol, and air the deposition for 30 min but not aired thoroughly;

5) Dissolve the deposition in 200 μl TE at 55° C. for 2 hours to dissolve DNA completely;

6) Polymerase chain reaction (PCR) system:

| | | |
|---|---|---|
| 10× PCR Buffer | 2 | μl |
| MgCL₂ (25 mM) | 1 | μl |
| dNTP mixture (10 mM, substrates) | 0.4 | μl |
| Upstream primer (50 μM) | 0.2 | μl |
| Downstream primer (50 μM) | 0.2 | μl |
| Template cDNA | 1 | μl |
| Taq DNA Polymerase | 0.2 | μl |
| ddH₂O | 15 | μl |
| Total volume | 20 | μl |

7) The electrophoresis of PCR products is shown in FIG. 1.

2. Animal Group and Administration

The positive mice at 1.5 months old are distributed by weight and cardiac function uniformly into the model group and the ginsenoside Rb1 administration group according to the echocardiography result, and each group comprises twelve mice. Ginsenoside Rb1, 75 mg/kg (State Yao zhun Zi, Yun Nan Tean Na Pharmaceutical Co., Ltd) is put into animal drinking water, and continuously administered for seven months.

3. Survival Analysis

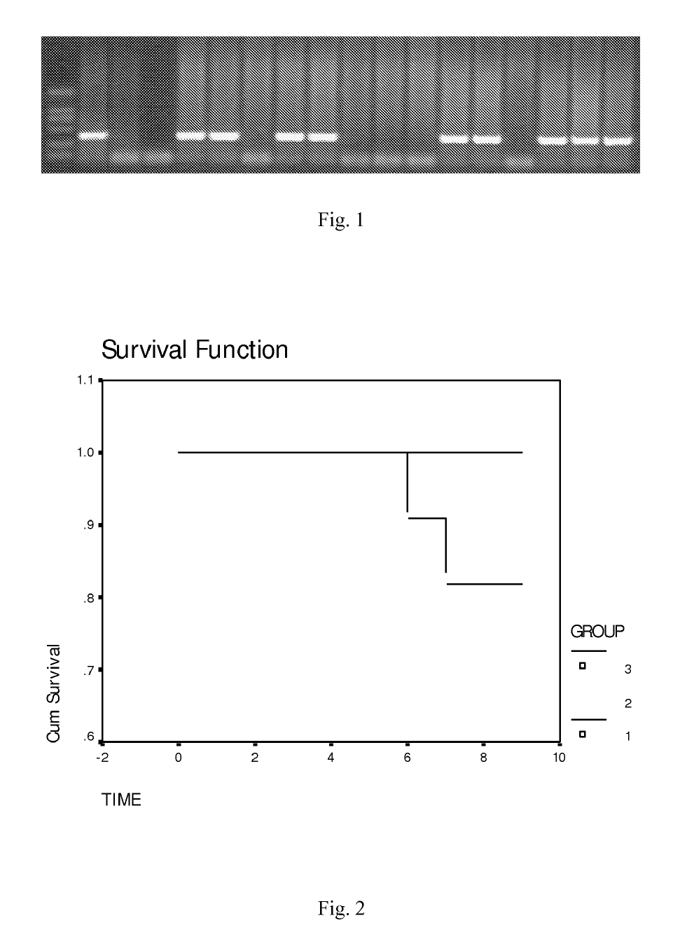

Individual death occurs in the feeding process of the cTnT^{R141W} transgenic mice model. From the beginning of administration to the observation endpoint, the death of animal of each group is recorded. The death of animal of the negative control group is zero, and the model group is four, and the ginsenoside Rb1 group is two. Analyzing the survival by the spss 10.0 statistical software, the results are shown in FIG. 2 which shows Rb1 could reduce the mortality rate of the cTnT^{R141W} dilated cardiomyopathy mice model.

### Example 2

Effects on the cardiac function and morphous of the cTnT^{R141W} mice model after a long-term administration of ginsenoside Rb1 (ultrasound)

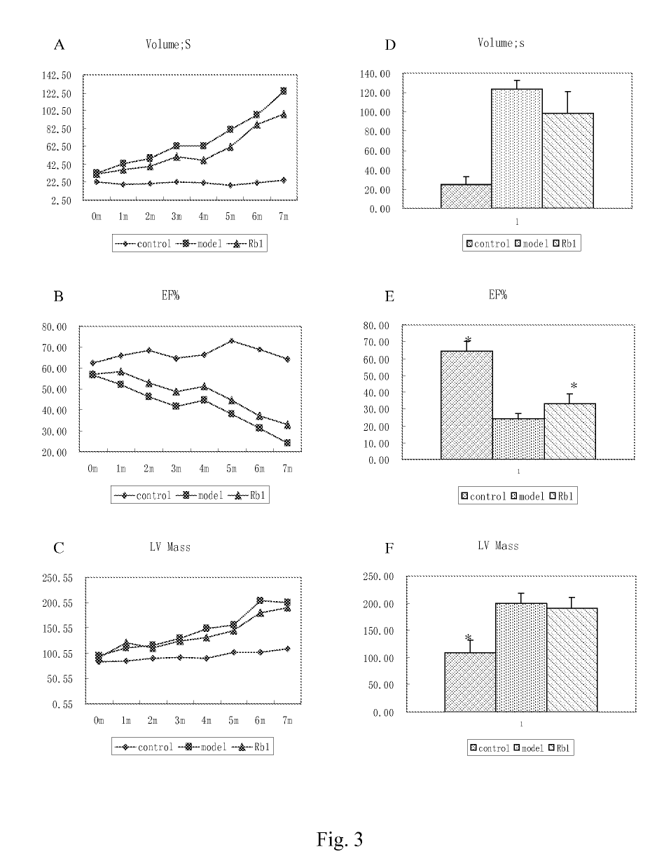

Echocardiography detection is performed once a month. Mice were anesthetized with tribromoethanol, scanned by small animal ultrasonic apparatus (Vevo 770 small animal ultrasonic detection system, Canada), with 30 MHz probe, M-mode echocardiography were analyzed: Body weight (BW), LVPW, d (the thickness of the left ventricular posterior wall at the end of the diastole), LVPW, s (the thickness of the left ventricular posterior wall at the end of the systole), LVAW, d (the thickness of the left ventricular anterior wall at the end of the diastole), LVAW, s (the thickness of the left ventricular anterior wall at the end of the systole), LV Mass (the weight of the left ventricular), Diameter, s (the inner diameter of the left ventricular at the end of the systole), Diameter, d (the inner diameter of the left ventricular at the end of the diastole), Volume, s (the volume of the left ventricular at the end of the systole), Volume, d (the volume of the left ventricular at the end of the diastole), SV (Stroke Volume), EF (Ejection Fraction), FS (Fractional Shortening) and CO (Cardiac Output). The result shows that Ginsenoside Rb1 can significantly reduce the dilation of cardiac chamber of the dilated cardiomyopathy mice model, and improve the cardiac function. Results are shown in Table 1 and FIG. 3.

TABLE 1
The Echocardiographic Parameters After Seven Months of Administration

| | 0 month | | | 1 month | | |
|---|---|---|---|---|---|---|
| | Control | Model | Rb1 | Control | Model | Rb1 |
| | n = 12 | n = 12 | n = 12 | n = 12 | n = 12 | n = 12 |
| BW | 21.69 ± 0.54 | 21.63 ± 2.60 | 22.72 ± 3.57 | 24.57 ± 1.27 | 24.80 ± 1.76 | 25.70 ± 1.41 |
| LVP | 0.60 ± 0.05 | 0.67 ± 0.09 | 0.67 ± 0.07 | 0.63 ± 0.10 | 0.64 ± 0.11 | 0.67 ± 0.06 |
| W; d | | | | | | |
| LVP | 0.84 ± 0.07 | 0.90 ± 0.12 | 0.85 ± 0.06 | 0.87 ± 0.16 | 0.85 ± 0.12 | 0.95 ± 0.11* |
| W; s | | | | | | |
| LVA | 0.77 + 0.16 | 0.67 + 0.07 | 0.67 + 0.06 | 0.79 + 0.08* | 0.71 + 0.04 | 0.84 + 0.12* |
| W; d | | | | | | |
| LVA | 1.09 ± 0.22 | 1.05 ± 0.14 | 0.99 ± 0.12 | 1.08 ± 0.11 | 1.11 ± 0.12 | 1.21 ± 0.13 |
| W; s | | | | | | |
| LV | 82.90 ± 18.99 | 95.36 ± 20.59 | 92.06 ± 17.12 | 84.24 ± 10.09* | 111.04 ± 22.14 | 120.38 ± 13.83* |
| Mass | | | | | | |
| (AW) | | | | | | |
| Diameter; s | 2.50 ± 0.25* | 2.88 ± 0.40 | 2.84 ± 0.41 | 2.38 ± 0.21* | 3.26 ± 0.35 | 3.01 ± 0.46 |
| Diameter; d | 3.73 ± 0.24* | 4.08 ± 0.30 | 4.02 ± 0.32 | 3.70 ± 0.17* | 4.45 ± 0.33 | 4.33 ± 0.38 |
| Volume; s | 22.59 ± 5.70* | 32.64 ± 10.55 | 31.51 ± 11.53 | 20.08 ± 4.66* | 43.37 ± 11.70 | 36.68 ± 13.77 |
| Volume; d | 59.64 ± 8.99* | 73.92 ± 12.39 | 71.50 ± 13.77 | 58.34 ± 6.52* | 90.10 ± 16.05 | 85.37 ± 17.55 |
| SV | 37.04 ± 4.48 | 41.29 ± 4.89 | 40.00 ± 4.42 | 38.26 ± 3.08* | 46.73 ± 8.30 | 48.69 ± 5.08 |
| EF | 62.50 ± 4.82 | 56.85 ± 8.31 | 57.05 ± 7.87 | 65.87 ± 4.58* | 52.27 ± 6.70 | 58.32 ± 7.31* |
| FS | 33.22 ± 3.40 | 29.71 ± 5.47 | 29.77 ± 5.07 | 35.66 ± 3.39* | 26.78 ± 4.12 | 30.78 ± 4.73* |
| CO | 17.04 ± 2.28 | 18.87 ± 2.94 | 19.59 ± 2.99 | 17.47 ± 2.11* | 22.31 ± 3.91 | 22.14 ± 2.64 |

| | 2 month | | | 3 month | | |
|---|---|---|---|---|---|---|
| | Control | Model | Rb1 | Control | Model | Rb1 |
| | n = 12 | n = 11 | n = 12 | n = 12 | n = 11 | n = 12 |
| BW | 26.84 ± 1.07 | 27.10 ± 1.76 | 26.77 ± 0.95 | 27.00 ± 1.10 | 26.84 ± 1.59 | 27.83 ± 1.74 |
| LVP | 0.60 ± 0.05* | 0.73 ± 0.11 | 0.69 ± 0.08 | 0.66 ± 0.07 | 0.67 ± 0.10 | 0.71 ± 0.06 |
| W; d | | | | | | |
| LVP | 0.90 ± 0.10 | 0.87 ± 0.12 | 0.88 ± 0.12 | 0.91 ± 0.09 | 0.85 ± 0.11 | 0.88 ± 0.11 |
| W; s | | | | | | |
| LVA | 0.78 + 0.10* | 0.67 + 0.04 | 0.78 + 0.05* | 0.81 + 0.11 | 0.73 + 0.08 | 0.75 + 0.08 |
| W; d | | | | | | |
| LVA | 1.18 ± 0.17* | 0.95 ± 0.12 | 1.13 ± 0.14* | 1.16 ± 0.13 | 1.09 ± 0.12 | 0.98 ± 0.09* |
| W; s | | | | | | |
| LV | 90.06 ± 18.49* | 115.29 ± 22.14 | 111.52 ± 7.92 | 92.61 ± 9.50* | 129.80 ± 16.27 | 124.88 ± 20.30 |
| Mass | | | | | | |
| (AW) | | | | | | |
| Diameter; s | 2.42 ± 0.27* | 3.44 ± 0.35 | 3.15 ± 0.39 | 2.53 ± 0.20* | 3.81 ± 0.26 | 3.47 ± 0.48* |
| Diameter; d | 3.89 ± 0.19* | 4.47 ± 0.33 | 4.31 ± 0.27 | 3.88 ± 0.27* | 4.78 ± 0.20 | 4.61 ± 0.35 |
| Volume; s | 20.97 ± 5.77* | 49.58 ± 11.70 | 40.29 ± 11.15 | 23.18 ± 4.47* | 62.63 ± 10.18 | 51.42 ± 15.53* |
| Volume; d | 65.58 ± 7.51 | 91.79 ± 16.05 | 83.96 ± 12.12 | 65.36 ± 10.48* | 106.54 ± 10.60 | 98.37 ± 16.57 |
| SV | 44.61 ± 3.09 | 42.21 ± 8.30 | 43.68 ± 2.20 | 42.18 ± 6.75 | 43.90 ± 2.34 | 46.95 ± 6.12 |
| EF | 68.46 ± 5.49* | 46.29 ± 6.70 | 52.93 ± 7.59* | 64.58 ± 3.20* | 41.52 ± 3.96 | 48.85 ± 9.39* |
| FS | 37.88 ± 4.23* | 23.12 ± 4.12 | 27.23 ± 5.02* | 34.84 ± 2.39* | 20.37 ± 2.19 | 24.88 ± 5.88* |
| CO | 20.14 ± 0.79 | 19.83 ± 3.91 | 21.89 ± 1.78 | 19.24 ± 2.82 | 20.83 ± 1.47 | 22.72 ± 3.18 |
| | 4 month | | | 5 month | | |
| | Control | Model | Rb1 | Control | Model | Rb1 |
| | n = 12 | n = 11 | n = 12 | n = 12 | n = 10 | n = 12 |
| BW | 28.86 ± 0.90 | 28.20 ± 1.77 | 28.72 ± 1.55 | 30.63 ± 1.06 | 29.27 ± 2.18 | 29.63 ± 1.25 |
| LVP | 0.65 ± 0.08 | 0.74 ± 0.10 | 0.71 ± 0.06 | 0.62 ± 0.07 | 0.71 ± 0.10 | 0.74 ± 0.06 |
| W; d | | | | | | |
| LVP | 0.96 ± 0.14 | 0.93 ± 0.12 | 0.89 ± 0.12 | 1.06 ± 0.07* | 0.79 ± 0.09 | 0.82 ± 0.08 |
| W; s | | | | | | |
| LVA | 0.75 ± 0.05 | 0.80 ± 0.07 | 0.81 ± 0.06 | 0.79 ± 0.09 | 0.81 ± 0.07 | 0.82 ± 0.12 |
| W; d | | | | | | |
| LVA | 1.17 ± 0.11 | 1.08 ± 0.09 | 1.17 ± 0.11* | 1.26 ± 0.23* | 1.09 ± 0.07 | 1.13 ± 0.10 |
| W; s | | | | | | |
| LV | 89.42 ± 18.86* | 149.46 ± 17.48 | 131.15 ± 12.93* | 101.46 ± 13.28* | 156.07 ± 28.05 | 150.86 ± 25.12 |
| Mass | | | | | | |
| (AW) | | | | | | |
| Diameter; s | 2.46 ± 0.34* | 3.82 ± 0.19 | 3.38 ± 0.35* | 2.31 ± 0.19* | 4.25 ± 0.36 | 3.86 ± 0.32* |
| Diameter; d | 3.85 ± 0.26* | 4.91 ± 0.18 | 4.56 ± 0.26* | 3.95 ± 0.15* | 5.21 ± 0.30 | 4.93 ± 0.30* |
| Volume; s | 22.10 ± 6.42* | 63.10 ± 7.81 | 47.56 ± 11.76* | 18.63 ± 3.83* | 81.51 ± 15.98 | 64.99 ± 12.65* |
| Volume; d | 64.46 ± 9.90* | 113.79 ± 9.87 | 95.93 ± 13.01* | 68.29 ± 6.08* | 130.65 ± 17.75 | 115.02 ± 16.33* |
| SV | 42.36 ± 5.44* | 50.68 ± 3.86 | 48.37 ± 3.32 | 49.65 ± 4.10 | 49.14 ± 7.23 | 50.03 ± 5.63 |
| EF | 66.30 ± 7.18* | 44.65 ± 2.97 | 51.07 ± 6.01* | 72.91 ± 4.09* | 37.95 ± 5.74 | 43.83 ± 3.93* |
| FS | 36.34 ± 5.92* | 22.23 ± 1.73 | 26.05 ± 3.64* | 41.64 ± 3.65* | 18.53 ± 3.18 | 21.76 ± 2.21* |
| CO | 18.81 ± 3.03* | 24.91 ± 2.03 | 24.42 ± 1.52 | 23.83 ± 3.03 | 23.41 ± 3.03 | 23.18 ± 3.13 |
| | 6 month | | | 7 month | | |
| | Control | Model | Rb1 | Control | Model | Rb1 |
| | n = 12 | n = 12 | n = 12 | n = 12 | n = 9 | n = 12 |
| BW | 31.80 ± 1.03 | 31.30 ± 2.04 | 32.00 ± 1.31 | 32.36 ± 1.11 | 32.10 ± 1.78 | 32.86 ± 1.28 |
| LVP | 0.61 ± 0.03* | 0.88 ± 0.09 | 0.80 ± 0.01* | 0.76 ± 0.08 | 0.73 ± 0.10 | 0.82 ± 0.13 |
| W; d | | | | | | |
| LVP | 1.03 ± 0.13* | 0.90 ± 0.11 | 0.93 ± 0.06 | 1.03 ± 0.08* | 0.82 ± 0.11 | 0.87 ± 0.19 |
| W; s | | | | | | |
| LVA | 0.80 ± 0.09 | 0.80 ± 0.07 | 0.74 ± 0.04* | 0.79 ± 0.07 | 0.77 ± 0.04 | 0.79 ± 0.12 |
| W; d | | | | | | |
| LVA | 1.27 ± 0.15* | 1.06 ± 0.05 | 1.09 ± 0.09 | 1.23 ± 0.11* | 0.96 ± 0.14 | 1.08 ± 0.17 |
| W; s | | | | | | |
| LV | 102.85 ± 11.70* | 204.14 = 27.57 | 180.65 ± 5.80* | 109.09 ± 23.38* | 199.84 ± 17.65 | 190.72 ± 20.29 |
| Mass | | | | | | |
| (AW) | | | | | | |
| Diameter; s | 2.45 ± 0.29* | 4.59 ± 0.23 | 4.38 ± 0.19 | 2.59 ± 0.34* | 5.10 ± 0.15 | 4.60 ± 0.44* |
| Diameter; d | 3.96 ± 0.18* | 5.40 ± 0.18 | 5.35 ± 0.19 | 3.95 ± 0.32* | 5.75 ± 0.18 | 5.46 ± 0.34* |
| Volume; s | 21.97 ± 5.89* | 97.40 = 11.01 | 87.21 ± 8.99 | 25.02 ± 8.18* | 123.93 ± 8.43 | 98.58 ± 22.36* |
| Volume; d | 68.71 ± 7.00* | 141.64 = 11.06 | 138.41 ± 11.40 | 68.37 ± 13.38* | 163.26 ± 11.94 | 145.92 ± 21.42* |
| SV | 46.74 ± 4.66 | 44.23 = 6.74 | 51.20 ± 5.31* | 43.35 ± 6.81 | 39.33 ± 7.17 | 47.35 ± 6.92* |
| EF | 68.62 ± 6.90* | 31.32 = 4.88 | 37.03 ± 3.07* | 63.97 ± 5.99* | 24.01 ± 3.32 | 33.02 ± 6.64* |
| FS | 38.39 ± 5.60* | 14.97 = 2.58 | 18.01 ± 1.71* | 34.52 ± 4.35* | 11.25 ± 1.67 | 15.92 ± 3.53* |
| CO | 20.11 ± 2.58 | 21.60 = 2.62 | 25.99 ± 2.23* | 22.19 ± 4.36 | 20.14 ± 3.84 | 24.35 ± 4.80* |

### Example 3

Effects on the cardiac microstructure of the mice model after a long-term administration of ginsenoside Rb1 (HE and Masson staining)

1. Method

1) Fix the heart and treated in a standard pathological procedure.

2) HE Staining;

3) Composite staining solution for 5 min;

4) Slightly washed by 0.2% acetic acid solution;

5) 5% phosphotungstic acid for 5-10 min;

6) Immersion cleaning twice by 0.2% acetic acid solution;

7) Brilliant green staining solution for 5 min, immersion cleaning twice by 0.2% acetic acid solution;

8) Ethanol dehydration, xylene transparent and sealed with neutral gum;

9) Observed under light microscope, collagen fibers show green, muscle fibers show red and red blood cells show nacarat.

2. Results

Observed under light microscope, myocytes showed uneven hypertrophy, disordered arrangement and some myocytes were degeneration with vacuolar; myocardial nucleuses were large, special shaped and deeply stained in the model group. While in the Ginsenoside Rb1 group, the hypertrophy and disordered arrangement of the myocytes were improved and the proliferation of interstitial collagen is also significantly decreased.

### Example 4

Effects on the left ventricular ultrastructure of the mice model with dilated cardiomyopathy after a long-term administration of ginsenoside Rb1 (electron microscopy)

1. Method

Fresh myocardial tissues are fixed with 2.5% glutaraldehyde solution, then post-fixed by 1% osmic acid, embedded by Epon812 after the acetone gradient dehydration, wherein the embedded number is five Epon812 per case, ultra-thin sections are double stained by uranyl acetate and lead citrate, and observing by JEM1230 type projection electron microscope. The apparatuses are JEM1230 type projection electron microscope (Japanese Electronics Company), LEICA UCT type microtome (Germany Leica Company).

2. The Preparation of Stationary Liquid

The preparation of the phosphate buffer (0.2M) is shown as follows.

The first liquid: 71.64 g Na₂HPO₄.12H₂O to be dissolved into 1000 ml ddH₂O

The second liquid: 31.21 g NaH₂PO₄.2H₂O to be dissolved into 1000 ml ddH₂O

The preparation of 0.2M phosphate buffer (pH=7.2, 50 ml): the first liquid 36.0 ml+the second liquid 14.0 ml

Glutaraldehyde stationary liquid in 0.1M phosphate buffer:

| | |
|---|---|
| 0.2M phosphate buffer | 50 ml |
| 25% glutaraldehyde | 10 ml |
| ddH₂O | 40 ml |
| Total volume | 100 ml |

3. Result

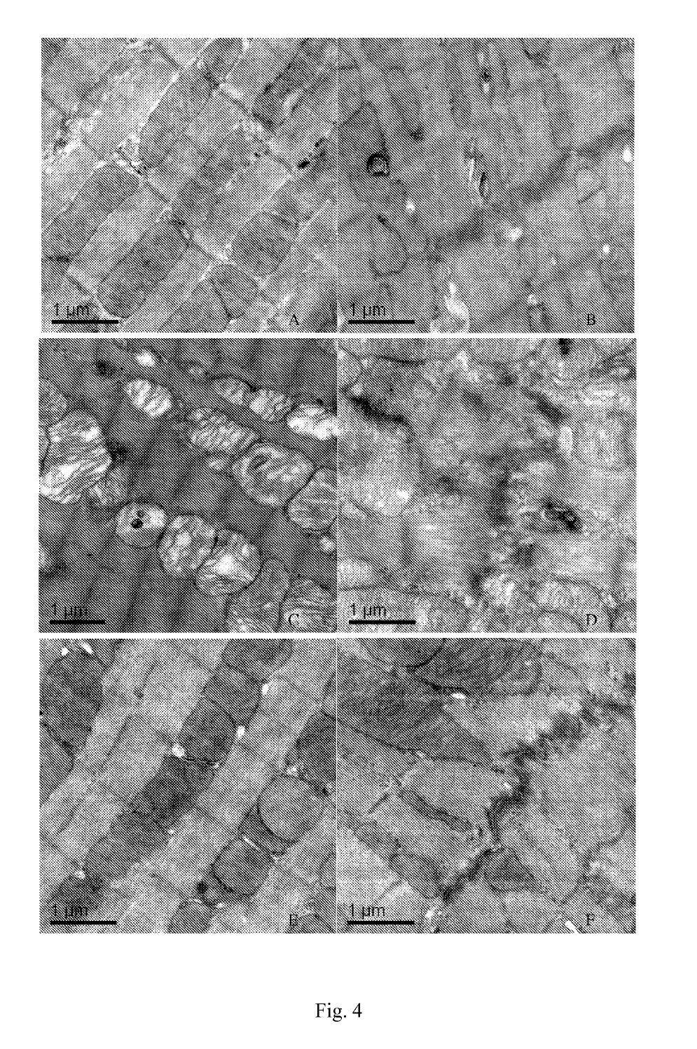

Myocytes show uneven thickness, while mitochondria show highly swelled, vacuoles, and sarcoplasmic reticulum moderately expanded and intercalated disc connections are destroyed in the model group. Myocytes of ginsenoside Rb1 group show clear structures, and most mitochondrial structures are normal, and sarcoplasmic reticulum slightly dilated, and intercalated disc structure are clear. The results are shown in FIG. 4.

### Example 5

Effects on some cardiac gene expression of the mice model with dilated cardiomyopathy after a long-term administration of ginsenoside Rb1 (RT-PCR method)

1. The Extraction of RNA

1) Pulverize the tissue in liquid nitrogen, and then add 1 ml Trizol liquid into the 50-100 mg tissue;

2) Place the grinded liquid at RT for 5 min, add 0.2 ml chloroform into the grinded liquid, tightly cap the centrifuge tube, and violently vortex the centrifuge tube for 15 sec;

3) Remove 0.5 ml the upper transparent liquid into a new centrifuge tube, add 0.5 ml isopropanol, placed at RT for 10 min, and centrifugate at 4° C., 12000 g for 10 min;

4) Remove the supernatant, add 1 ml 75% ethanol and centrifugate at 4° C., 7500 g for 5 min;

5) Carefully remove the supernatant, then dried at RT for 5-10 min, and then dissolved in DEPC-treated water.

2. RT-PCR (Reverse Transcription—Polymerase Chain Reaction)

| | |
|---|---|
| 1) 10 mM dNTP mix | 1 μl |
| 50 μM oligo(dT)₂₀ | 1 μl |
| RNA | 5 μl |
| DEPC-treated water | 3 μl |
| Total volume | 10 μl |

at 65° C. for 5 min, then placed on ice for over 1 min.

| | |
|---|---|
| 2) 10× RT buffer | 2 μl |
| 25 mM MgCl₂ | 4 μl |
| 0.1M DTT | 2 μl |
| RNaseOUT | 1 μl |
| Total volume | 9 μl |

Add the above mixture into the tube in step 1), then incubated at 42° C. for 2 min;

3) Add 1 μl SuperScript, II RT at 42° C. for 50 min;

4) Placed on ice for 15 min;

5) Add 1 μl RNase H, then incubated at 37° C. for 20 min;

6) PCR reaction system is shown as follow:

| | | |
|---|---|---|
| 10× Buffer | 2 | μl |
| 25 mM MgCl₂ | 1 | μl |
| 10 mM dNTP mixture | 0.4 | μl |
| Primer sense | 0.2 | μl |
| Primer antisense | 0.2 | μl |
| Template cDNA | 1 | μl |
| Taq Polymerase | 0.2 | μl |
| ddH₂O | 15 | μl |
| Total volume | 20 | μl |

7) General conditions of PCR are shown as follow. Pre-denaturation at 94° C. for 30 min, and extension at 72° C. for 30 min And the annealing temperature, the annealing time, the primer sequences and the cyclic numbers are shown in Table 2.

TABLE 2
RT-PCR primer sequences and reaction conditions

| | | | | Annealing | | |
|---|---|---|---|---|---|---|
| | | Consensus | Product | Tempera- | | Cycle |
| Gene | Primer Sequences(5′-3′) | Sites | Length | ture | Time | Numbers |
| Gapdh | F: TCGGTGTGAACGGATTTGGC | S60-80 | 320 | 60 | 30 | 25 |
| | R: CAAGTGGGCCCCGGCCTTCT | A360-380 | | | | |
| Cx40 | F: AGGAGGAAAGGAAGCAGAAGG | S54-74 | 148 | 55 | 30 | 25 |
| | R: GCGGAAAATGAACAGGACAGTGAG | A179-202 | | | | |
| Cx43 | F: AAGTGAAAGAGAGGTGCCCAGAC | S210-232 | 173 | 55 | 30 | 23 |
| | R: CAGACTGTTCATCACCCCAAGC | A362-383 | | | | |
| Ecad | F: AGTACATCCTCTATTCTCATGCCGT | S813-837 | 157 | 60 | 30 | 26 |
| | R: GGTTCCTGGAACAGCGCC | A953-970 | | | | |
| Ncad | F: AGGTGGAGGAGAAGAAGACCAGGACT | S2983-3008 | 204 | 55 | 30 | 25 |
| | R: GTCAGCAGCTTTAAGGCCCTCATT | A3164-3187 | | | | |
| Pcad | F: AGGACCAGGACTATGACATCACCCA | S2170-2194 | 161 | 60 | 30 | 28 |
| | R: TCAGGTTCTCGATGATGAAGTTCCC | A2307-2331 | | | | |
| Itga8 | F: GATATAAAGCCTGCTGCCTCC | S2764-2784 | 364 | 54 | 30 | 25 |
| | R: ACGTCTTAACCGCTGTGCTCC | A3107-3127 | | | | |
| Itgbp | F: ACAGTGGGACGTGCTTGAGTC | S217-237 | 362 | 54 | 30 | 25 |
| | R: TGCCATCTAAATAGACCACTTC | A557-578 | | | | |
| Fgf2 | F: CGACCCACACGTCAAAC | S362-378 | 267 | 62 | 30 | 38 |
| | R: CACTTAGAAGCCAGCAGC | A464-483 | | | | |
| Fgf7 | F: CCAAACAGAACAAAAGTCAAG | S66-86 | 571 | 52 | 30 | 38 |
| | R: GATTTCCATGATGTTGTAGC | A627-647 | | | | |
| Fgf8 | F: TGCCTGCTGTTGCACTTG | S227-244 | 235 | 60 | 30 | 38 |
| | R: TGTCGCTGTGTGACTTTAG | A444-462 | | | | |
| Fgf10 | F: TCCGTACAGTGTCCTGGAGATA | S671-692 | 130 | 60 | 30 | 38 |
| | R: AGTCGTTGTTAAACTCTTTTGAG | A779-801 | | | | |

wherein, Gapdh serves as the loading control.

Cx40, Cx43: connexin 40, connexin 43;

E-, P-, N-cad: E-, P-, N-cadherin;

Itga8: Integrin alpha 8;

Itgbp: integrin beta 1 binding protein 3;

FGF 2, 7, 8, 10: fibroblast growth factor 2, 7, 8, 10.

3. Results

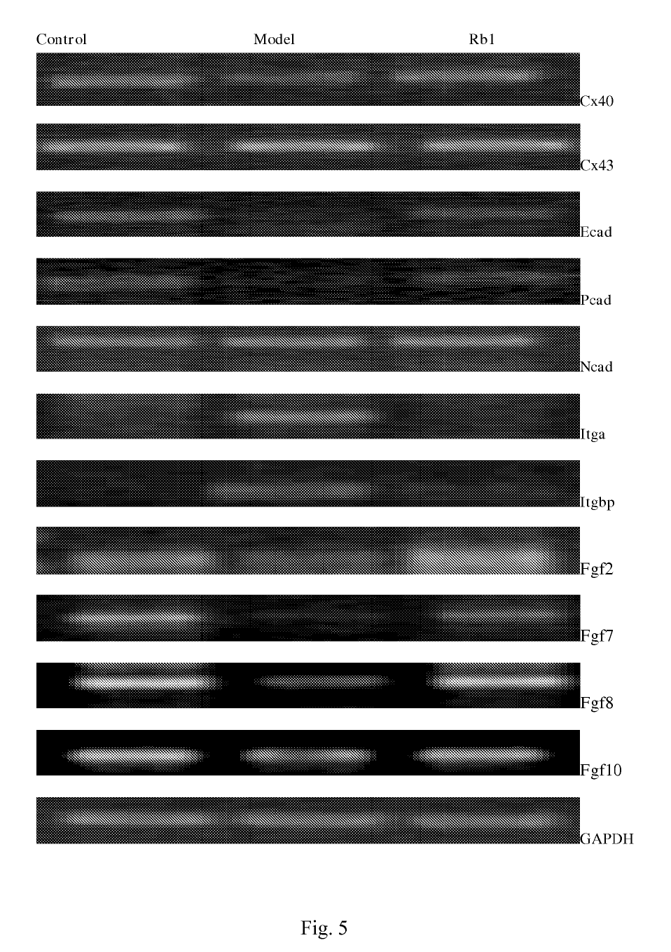

Ginsenoside Rb1 can increase the expression of Cx40, E-cadherin and P-cadherin, decrease the expression of Itga8 and Itgbp, and improve the expression of the fibroblast growth factor FGF. The results are shown in FIG. 5.

### INDUSTRIAL APPLICABILITY

The present invention shows some advantages as follow.

The present invention was screened from twenty monomer components of traditional Chinese medicine which show low toxicity, clear structure and potential therapeutic effects on cardiovascular system using the cTnT^{R141W} transgenic mice model. The results show that ginsenoside Rb1 has the prevention effects on dilated cardiomyopathy. Long-term administration of ginsenoside Rb1 can reduce the mortality rate, improve the cardiac function, reduce the dilation of the left ventricular, reduce the hypertrophy of myocytes and the proliferation of collagen, reduce the uneven thickness of myocytes, mitochondria swelling, break of crista, sarcoplasmic reticulum expansion and destruction of intercalated disc connection, increase the expression of connexin Cx40, E-cadherin and P-cadherin, decrease the expression of Itga8 and Itgbp, and improve the expression of the growth factor FGF2, FGF7, FGF8 and FGF10.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

1. A method of preparing medicaments for treating dilated cardiomyopathy comprising the step of applying ginsenoside Rb1.

2. The method, as recited in claim 1, wherein ginsenoside Rb1 comprises ginsenoside Rb1 monomer and excipient or carrier.

3. The method, as recited in claim 1, wherein the dilated cardiomyopathy is the death caused by R141W mutation in cTnT protein.

4. The method, as recited in claim 1, wherein the dilated cardiomyopathy is the progressive dilation of the left ventricular and progressive decrease of cardiac function caused by R141W mutation in cTnT protein.

5. The method, as recited in claim 1, wherein the dilated cardiomyopathy is irregular hypertrophy of left ventricular myocytes and the proliferation of interstitial collagen caused by R141W mutation in cTnT protein.

6. The method, as recited in claim 1, wherein the dilated cardiomyopathy is uneven thickness and disorderly arrangement of myocardial fibers, mitochondria swelling, cristae fragmentation, sarcoplasmic reticulum expansion and intercalated disk connections failure caused by R141W mutation in cTnT protein.

7. The method, as recited in claim 1, wherein the dilated cardiomyopathy is the decreased expression of Cx40, and the increased expression of E-cadherin, N-cadherin, Itga8 and Itgbp caused by R141W mutation in cTnT protein.

8. The method, as recited in claim 1, wherein the dilated cardiomyopathy is the decreased expression of FGF2, FGF7, FGF8 and FGF10 caused by R141W mutation in cTnT protein.

9. The method, as recited in claim 1, wherein ginsenoside Rb1 is used in the form of drug combination.

10. The method, as recited in claim 1, wherein ginsenoside Rb1 is used in the form of monomer.
